(19) United States
(12) Patent Application Publication
(10) Pub. No.: US 2025/0302355 A1
(43) Pub. Date: Oct. 2, 2025

(54) Method of teaching and enabling use of the HAIL ™ process

(71) Applicant: Linda Rossetti, Winchester, MA (US)

(72) Inventor: Linda Rossetti, Winchester, MA (US)

(21) Appl. No.: 19/096,076

(22) Filed: Mar. 31, 2025

Related U.S. Application Data
(60) Provisional application No. 63/572,880, filed on Apr. 1, 2024.

(51) Int. Cl. A61B 5/16; A61B 5/00

(52) U.S. Cl. CPC A61B 5/165; A61B 5/7435; A61B 5/7475

(57) ABSTRACT

Methods and apparatus that improve the capabilities of a subject to respond differently to the emotions that accompany conflict and major change. The methods and apparatus facilitate or assistant with the performance of the HAIL™ method, an accessible repeatable process that creates in a subject a new context for emotions and improve the subject's ability to understand, process, and alter behaviors related to their emotions. A graphical user interface (GUI) can be used to guide a subject through the steps of the HAIL™ method by displaying prompts and receiving responses to those prompts. The graphical user interface (GUI) may be used by the subject performing the HAIL™ method or a practitioner assisting the aforementioned subject.

### RELATED APPLICATION

This application claims the benefit of U.S. Provisional Application No. 63/572,880, filed on Apr. 1, 2024. The entire teachings of the above application are incorporated herein by reference.

### BACKGROUND

Conflict and change often trigger undesirable and unproductive reactions and thought patterns in individuals without the proper tools to experience them. The ability of people to self-reflect on and improve their reactions and responses to periods of conflict and transition is severely lacking. This limits their capacity for developmental growth both internally and externally. While every adult possesses the capacity for developmental growth, few successfully achieve it. One study conducted by researchers at Harvard's Graduate School of Education found that the majority of U.S. college educated adults, or 59%, never fully achieves a developmental accomplishment, while only 7% fully exploit opportunities provided for progression. Other studies estimated even lower levels of developmental growth, indicating a large unmet need for methods to facilitate widespread improvement in how people process, understand, and react to challenging circumstances.

Furthermore, psychologists and medical practitioners have observed an increasing number of patients suffering from anxiety, panic, and similar symptoms that were not responding to traditional treatments such as psychological therapy or psychiatric medication. Cognitive Behavioral Therapy (CBT) has been found to be an effective treatment for these increasingly common ailments. CBT focuses on identifying and changing a patient's thinking patterns. For example, it may empower a patient to recognize an unhealthy coping mechanism, such as avoidance, understand the motivation for that mechanism, and subsequently provide alternative ways to respond to that motivation. Unfortunately, CBT often requires highly trained and specialized practitioners and frequently needs to be performed in a clinical environment. These restrictions create barriers that prevent widespread application of CBT and limit access for the growing number of individuals it would benefit. A need exists to be able to disseminate the latest science of human development and research reflected in and utilized by CBT and to empower individuals to understand, process, and reframe their emotions successfully outside a clinical environment.

### SUMMARY

HAIL™ is a 4-step mnemonic (Honor, Ask, Influence, Learn) technique that empowers its users to respond differently to the emotions that accompany conflict and major change. It is an accessible repeatable process that creates in the user a new context for emotions. HAIL™ utilizes and incorporates the newest advances and thinking in the field of human development, a multidisciplinary study that incorporates psychology, psychiatry, sociology, leadership, neurology, biology, and adult learning. The HAIL™ method empowers a user to more successfully respond to challenges related to shifts in their self-concept and defuses an emotion's power over the individual. The method enables the user to access more of their response capabilities (e.g., their untapped potential) in responding to a variety of challenges.

Embodiments of the technique include methods for a user practicing HAIL™ utilizing HAIL™ as a practitioner with a subject, and teaching practitioners and users how to apply HAIL™. The invention further includes apparatuses and computer-executable software that are utilized as part of the aforementioned methods.

A method of facilitating emotional reframing comprising obtaining, obtaining, a time period and identifying emotions experienced by a person during the time period. The method also includes receiving a selection of at least one emotion from the emotions experienced by the person during the time period and identifying a meaning held by the person associated with the selected at least one emotion. Subsequently, the method includes identifying behaviors of the person associated with the selected at least one emotion and identifying effects of the selected at least one emotion. The method facilitates improvement by the person to understand, process, or alter behaviors related to the emotions experienced by the person during the time period.

The time period may include a challenge experienced by the person.

In some embodiments, a practitioner performs or assists the person perform t least one of i) obtaining a time period, ii) identifying the emotions, iii) receiving the selection of at least one emotion, iv) identifying the meaning held by the person, v) identifying the behaviors of the person, and vi) identifying effects of the selected at least one emotion.

The method may further comprise generating a graphical user interface (GUI). The graphical user interface (GUI) configured to display at least one prompt element comprising prompts for use by the practitioner, and at least one input element configured to receive responses provided by the person.

The improvement by the person to understand, process, or alter behaviors related to the emotions experienced by the person during the time period can include at least one of an ability to handle failure, an ability to deal with difficulties, an ability to handle unpleasant feelings, an ability to be aware of multiple options, an ability to address new challenges.

An apparatus for facilitating a user's emotional reframing comprising a processor and one or more storage devices storing instructions that are operable, when executed by the processor, to generate a graphical user interface (GUI). The generated GUI is configured to display a first element configured to receive and display emotions experienced by a user during a time period, a second element configured to receive and display a meaning held by the user associated with at least one emotion selected from the emotions experienced by the user during the time period, a third element configured to receive and display behaviors of the user associated with the selected at least one emotion, and a fourth element configured to receive and display effects of the selected at least one emotion. The generated GUI facilitating improvement of the user to understand, process, or alter behaviors related to the emotions experienced by the user during the time period.

The processor and one or more storage devices may be one of a smartphone, computer, or tablet.

In some embodiments, at least one of the first element, the second element, the third element, and the fourth element comprises at least one prompt element configured to display, to the user, prompts and at least one input element configured to receive, from the user, responses to the displayed prompts. In such embodiments, the received responses to the displayed prompts can facilitate improvement of the user to understand, process, or respond differently to the emotions experienced by the user during the time period. In such embodiments, the graphical user interface (GUI) can be further configured to display a summary element displaying the received responses to the displayed prompts.

An apparatus for facilitating a user's emotional reframing comprising a processor and one or more storage devices storing instructions that are operable, when executed by the processor, to generate a graphical user interface (GUI). The generated GUI configured to display a prompt element configured to display conversational prompts, the conversational prompts configured to be utilized to enable a subject to: i) identify emotions experienced by the subject during a time period, ii) identify a meaning held by the subject associated with at least one emotion selected from the emotions experienced by the subject during the time period, iii) identify behaviors of the subject associated with the selected at least one emotion, and iv) identify effects of the selected at least one emotion. The generated GUI also configured to display at least one display element configured to display: i) the emotions experienced by the subject during the time period, ii) the at least one additional period of time the subject experienced the selected at least one emotion, iii) the behaviors of the subject associated with the selected at least one emotion, and iv) the effects of the selected at least one emotion. The generated GUI facilitating improvement of the subject to understand, process, and alter behaviors related to the emotions experienced by the subject during the time period.

A method of facilitating a user's emotional reframing through use of a processor and one or more storage devices configured to store instructions that are operable when executed by the processor to generate a graphical user interface (GUI). The method comprises receiving, from a user, and displaying, by a first element of the GUI, emotions experienced by the user during a time period. The method also including receiving, from the user, and displaying, by a second element of the GUI, at least one additional period of time the user experienced at least one emotion selected from the emotions experienced by the user during the time period. The method then involves, receiving, from the user, and displaying, by a third element of the GUI, behaviors of the user associated with the selected at least one emotion and receiving, from a user, and displaying, by a fourth element of the GUI, effects of the selected at least one emotion. The generated GUI facilitating improvement of the subject to understand and process the emotions experienced by the subject during the time period.

The generated graphical user interface (GUI) can be further configured to display at least one input element configured to receive responses to the conversational prompts. The at least one input element can be is configured to receive responses to the conversational prompts by text, audio, or visual methods. The at least one subject element may be further configured to display the received, responses to the conversational prompts by text, audio, or visual methods.

A method of facilitating a user's emotional reframing through use of a processor and one or more storage devices configured to store instructions that are operable when executed by the processor to generate a graphical user interface (GUI). The method comprises receiving and displaying, by a first element of the GUI, emotions experienced by the user during a time period and receiving and displaying, by a second element of the GUI, a meaning associated with at least one emotion selected from the emotions experienced by the user during the time period. The method further comprises receiving and displaying, by a third element of the GUI, behaviors of the user associated with the selected at least one emotion and receiving, and displaying, by a fourth element of the GUI, effects of the selected at least one emotion. The graphical user interface (GUI) facilitates improvement of a user to understand, process, or alter behaviors related to the emotions experienced by the user during the time period.

The graphical user interface (GUI) can be generated by a smartphone application.

In some embodiments, at least one of the first element, the second element, the third element, and the fourth element comprise at least one prompt element configured to display prompts and at least one input element configured to receive responses to the displayed prompts. In such embodiments, at least one of the emotions experienced by the user during a time period, a meaning associated with the at least one emotion selected from the emotions experienced by the user during the time period, behaviors of the user associated with the selected at least one emotion, and effects of the selected at least one emotion can comprise the received responses to the displayed prompts. The method may also include storing, in the one or more storage devices the received responses to the displayed prompts.

### BRIEF DESCRIPTION OF THE DRAWINGS

The foregoing will be apparent from the following more particular description of example embodiments, as illustrated in the accompanying drawings, in which like reference characters refer to the same parts throughout the different views. The drawings are not necessarily to scale, emphasis instead being placed upon illustrating embodiments.

FIG. 1 is a flow chart of an embodiment of the invention for facilitating emotional reframing utilizing the HAIL™ method.

FIG. 2 is a graphical user interface (GUI) utilized by an embodiment of the invention for facilitating a user's emotional reframing.

FIGS. 3a and 3b are a graphical user interface (GUI) utilized by an embodiment of the invention for facilitating guidance by a practitioner of a subject's emotional reframing.

FIG. 4 is a flow chart of an embodiment of the invention enabling practice of the HAIL™ method.

FIGS. 5a-g are screenshots of an application configured to generate and display a graphical user interface (GUI) utilized by an embodiment of the invention for facilitating a user's emotional reframing.

FIG. 6 illustrates a computer network or similar digital processing environment in which the present embodiments may be implemented.

FIG. 7 is a diagram of the internal structure of a computer (e.g., client processor/device or server computers) in the computer system of FIG. 5.

### DETAILED DESCRIPTION

A description of example embodiments follows.

One research survey found that over 90% of respondents lacked sufficient vocabulary to be able to describe and understand times of transition in their lives. These times were defined by circumstances during which one's thinking about their self-concept (an idea of the self constructed from the beliefs one holds about oneself and the responses of others) was challenged in some fashion, including, but not limited to, personal, professional, physical, spiritual, and other dimensions. A further 70% of respondents responded to uncertainty about themselves or their identity with reflexive, automatic responses (e.g., responses familiar to them). This can be undesirable because it includes uncritically replicating the past and/or demonstrates lack of ability to recenter or reestablish their self-concept following a period of instability or uncertainty related to one's self-concept. From a developmental standpoint, these limited and/or automatic responses result in most individuals stalling, disengaging, or retreating instead of progressing during times of instability, transition, and/or crisis. Increasing the severity of this problem is that over 60% of respondents also replied that they believed that they did not have a choice in how they responded to the challenging circumstances. This failure to recognize choice, demonstrated by thoughts such as “What choice do I have . . . ,” “I feel forced to do . . . ,” and “I have to do this, there are too many consequences . . . ” stunts emotional growth and artificially constrains a person's ability to respond to new, unexpected, or even repeated challenges or changes.

Techniques disclosed herein are designed to facilitate, teach, and/or assist with the application of the HAIL™ method. HAIL™ is an emotional reframing tool that allows users, either alone, in a group, or with the assistance of a practitioner, to understand, process, and, if desired, change their emotional and behavioral responses and reactions to emotions and/or circumstances. HAIL™ is especially helpful to people experiencing conflict and/or periods of change as it empowers its users to succeed at transformative change and even how its users approach and process change and/or conflict. This success can often be provided by understanding and controlling a user's past automatic responses and, through that control, provide awareness of alternative and/or additional behavior responses that would not have been otherwise available to the user. As a non-limiting example, the HAIL™ method can be utilized to empower students to respond differently to a variety of academic, social, and personal challenges. The HAIL™ method is designed to aid individuals by providing awareness of an additional and/or alternative behavioral response when one is faced with instability or uncertainty surrounding their self-concept. These instances of instability or uncertainty can be initiated by a wide variety of common experiences that occur over the arc of adulthood. The HAIL™ method can be used to counteract a person's incorrect view that their responses to challenging circumstances are limited, provide tools to broaden their available reactions, and deepen their understanding of their own emotional self-processing and reflexive responses.

The HAIL™ method focuses on enabling users' developmental growth by promoting their willingness and ability to act on their own purposes, values, feelings, and meanings, rather than those they have uncritically assimilated from others or their environment. To do so, a user can become aware of those assumptions upon which they have previously relied and address the emotional response created by that new awareness. To assist the user in addressing this emotional response, which can be intense and overwhelming, embodiments of the disclosed methods and apparatuses provide structure to guide a user, individually or with a practitioner, through the concrete series of steps set out by the HAIL™ method. The HAIL™ method helps the user address the intensely threatening emotional experience related to major change by aiding a user in reframing their emotions and gaining greater awareness of the reasons for and effect of those emotions. Therefore, the HAIL™ method enables the individual to navigate developmental progression at a greater rate than before-a critically important skill due to the transformational change occurring throughout society. The HAIL™ method also addresses a universal barrier to adult developmental growth by offering a method of reframing the intense emotional experience of instability or uncertainty that occurs repeatedly throughout adulthood.

The HAIL™ method facilitates and provides guidance for a user to a) externalize their emotions, b) observe and reflect on their relationship with their emotions, c) assess their emotions' roles in how they know themselves and the world, d) suspend judgment related to the presence of an emotion, e) defuse the need to respond to, categorize, or pathologize the emotion, and f) understand the influence of their emotions on their behavior. When the HAIL™ method is utilized by a practitioner with a user, it facilitates the practitioner to guide the user through proven science-based steps and create a holding environment for safety and reflection that empowers the user. Individuals can employ the HAIL™ method as both practitioner and users and apply their experience in either role to improve their ability to use, facilitate, or teach the HAIL™ method. The HAIL™ method, and the related embodiments of the methods and apparatuses described herein, can be applied in a variety of settings, including leadership development programs, employee wellness programs, team effectiveness activities, conflict resolution seminars, DOI (diversity, opportunity, and inclusion) initiatives, resiliency training, and workshops on problem solving, decision making, and negotiating. The HAIL™ method can also serve as conversation scaffolding, for a user's internal thought process or for an external discussion with a practitioner, that provides concrete and discrete steps to guide a user through the often difficult and taxing process of developmental growth.

The HAIL™ method and embodiments of the disclosed methods and apparatuses create positive outcomes for their users including, but not limited to, providing new context for a user's emotions and their relationship with those emotions, improving a user's ability to ask themselves new questions, and improving the capacity of a user to reset on many different levels, e.g., their emotions, their energy, their way of knowing themselves, their way of understanding others, and their way of knowing the world. As a further non-limiting example, when utilized by college students, the HAIL™ method and embodiments of the disclosed methods and apparatuses were able to 1) teach students a resilience-promoting framework that enables them to stay on track; 2) reorient challenges so that students can move forward with new perspectives; 3) increase the range of responses a student can apply to common challenges, including overcoming academic challenges, managing financial and living arrangements, and adapting to new social and environmental roles; 4) serve as an upstream intervention involving non-clinical partners who help students prevent the onset of mental health difficulties; 5) contribute to improvements in retention by reducing a student's propensity to pathologize feelings common to the college experience; 6) increase a student's agency or willingness to act on their own behalf; and 7) encourage interaction and belonging through connection with other individuals and with the greater campus and off-campus communities.

To begin, in an example application of the HAIL™ method, a user selects a time of major change or conflict in their career or life. Subsequently, the user applies the four steps of the HAIL™ method, each represented by a letter of the mnemonic.
1. Honor: Honor focuses on having the user identify and name the emotions that were active during the selected time. The term emotion includes, but is not limited to emotions, feelings, behaviors, sensations, and responses. The goal of this step is to create a list of emotions that are active for the user during the time they have chosen.
2. Ask: Ask is a deeper reflection on one of the emotions identified in the previous step. A user chooses one emotion from the list created in the Honor step and investigates the meaning that the emotion holds for them. A user may also identify other times they experienced the selected emotion to help them investigate its meaning. The goal of this step is to have the user explore the meaning that the selected emotion has in their history.
3. Influence: Influence focuses on how the selected emotion causes the person to act when it is/was being experienced. The goal for this step is for the user to observe and recognize their own behaviors, intentional and/or reflexive, that accompany the selected emotion.
4. Learn: Learn asks the user to reflect on the effects of the selected emotion, positive and negative, and to improve their understanding of how the emotion guides their actions and decision making, for example how the emotion allows the user to see, consider, understand or learn. The goal of this step is to have the user explore the lessons or wisdom that accompany the selected emotion, and the insights gained from the prior steps.

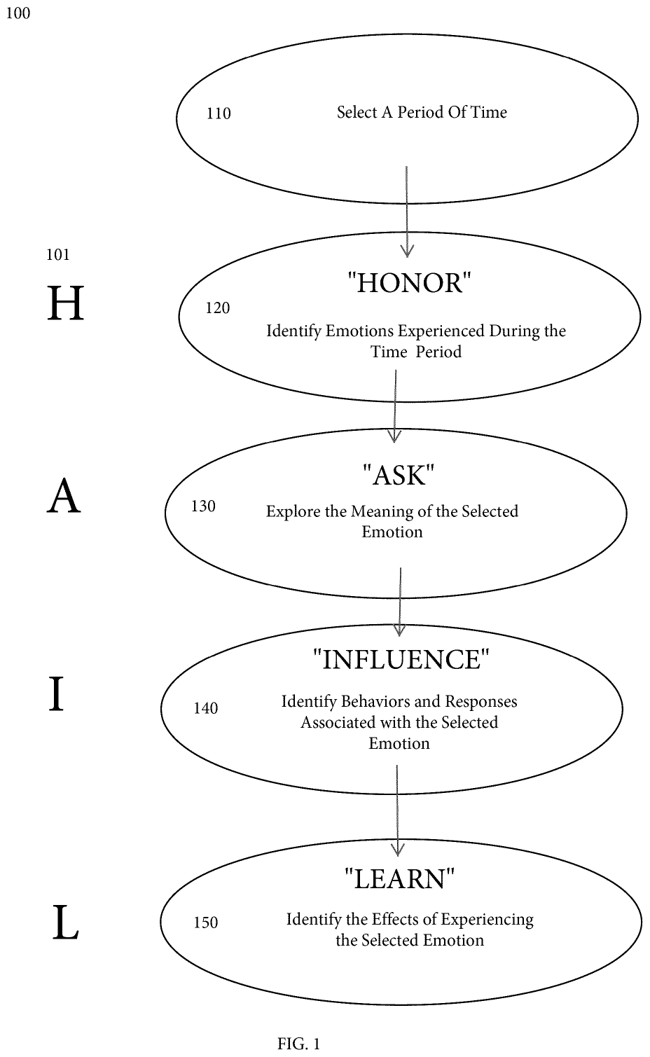

FIG. 1 is a flow chart 100 of an embodiment of the invention of facilitating emotional reframing utilizing the HAIL™ method. As discussed further below, the HAIL™ method may be practiced alone, with a practitioner, in a group setting, and/or utilizing apparatuses with specialized graphical user interfaces (GUIs). In step 110, a user or a subject selects a period of time. The period of time may be a time of transition, change, or conflict. The period of time may also be indicative of an emotion, reflexive response, or detrimental behavior that the user wishes to examine and/or change. The subsequent steps Honor 120, Ask 130, Influence 140, and Learn 150 comprise the HAIL 101 mnemonic to assist in memorization, repetition, and application.

In Honor 120, a user identifies emotions they experienced during the selected time period. The user may identify as many emotions as come to mind and/or be provided with lists of emotions to facilitate identification and selection. A user may write down their emotions, verbalize their emotions, record them digitally, or record them with the assistance of a practitioner or other user. The term “emotions” when utilized in the HAIL™ method and embodiments of the disclosed methods and apparatuses can also include reflective reactions, feelings, sensations, beliefs, thought patterns, and negative/positive effects such as, but not limited to, anxiety or contentment.

In Ask 130, a user explores the meaning of an emotion selected from those identified in the Honor 120 step. This can include identifying other time periods when the user experienced the selected emotion and reflecting on how they processed and expressed that emotion. The user investigates what meaning the emotion holds and how they experience the emotion in different circumstances and time periods. The user may also explore why the emotion is being experienced during the selected time period based on how it expresses itself in their past. The term “periods of time,” when utilized by the HAIL™ method and embodiments of the disclosed methods and apparatuses, encompasses not just specific temporal periods but also can include connected emotional feelings, abstract representations, and patterns of behavior/responses.

In Influence 140, a user identifies behaviors and responses associated with the selected emotion. This can be done by exploring how the user behaves or responds when they experience the emotions during the additional time periods identified during the Ask 130 step. During the Influence 140 step, a user will explore correlations between the selected emotions and their own behaviors and responses that accompany or are triggered by the emotion. The term “behaviors and responses” when utilized by the HAIL™ method and embodiments of the disclosed methods and apparatuses can further include secondary emotions, patterns of behavior, or barriers/constrictions on actions.

In Learn 150, a user identifies the effects of the selected emotion, including the effects of its associated behaviors and responses. The user is invited to explore the insights derived from the emotion and its presence, enabling the user to think differently about themselves and the circumstances that induced the emotion The user can apply what they have learned about the emotion, its effect, its meaning, and/or its associations to the period of time identified in step 110. The term “effects” when utilized by the HAIL™ method and embodiments of the disclosed methods and apparatuses can further include correlated or opposing emotions, lack of other emotions, induced or constrained responses, or other mental or behavioral responses.

As a result of the structured self-reflection and exploration techniques of the HAIL™ method described in flow chart 100, the user will be able to improve their understanding and processing of the emotions experienced during the selected time period. In particular, the user will be able to understand, expand, and control their behavioral and emotional responses to challenging circumstances, at least in part, because of an improved ability to understand and process the emotions experienced. This positive outcome of improving a user's ability to understand and process emotions can be referred to as emotional reframing and may include the ability of the user to identity and practice new behavioral responses and change/control previously reflexive responses.

While the HAIL™ method and embodiment of the invention can be performed by a user alone, they also provide useful tools and techniques for a practitioner to facilitate a subject's, or user's, emotional reframing. This can be especially useful as an upstream non-clinical intervention to address sub-acute mental health issues, to create a holding environment wherein individuals can safely explore new behavioral and emotional responses, and to create a framework to support others who are navigating challenges. Beneficially, the HAIL™ method and embodiment of the invention can be taught to non-expert practitioners and do not require extensive training in therapy or psychology. The incorporation of a practitioner does not alter the HAIL™ method set out in flow chart 101 and steps 110-150; the practitioner is empowered to assist the user in performing or subject the user to follow steps 110-150. This assistance can be provided through a series of prompts from the practitioner and/or conversations between the subject and the practitioner. As non-limiting examples, during Honor 120 the practitioner may ask the subject to identify emotions that are/were active for them during the selected time. During Ask 130 the practitioner may ask the subject to choose at least one emotion for their Honor list and explore what meaning the emotion holds in their experiences. During Influence 140 the practitioner may ask the subject to consider the behaviors that accompany the selected emotion and how that emotion influences them. During Learn 150, the practitioner may ask the subject to imagine that the selected emotion is active to help them learn or understand something important and what the subject might learn from the presence of the emotion and offer insights upon which the user can reestablish stability in their self-concept. Additional prompts and questions are available for the practitioner for all steps 120-150 to help assist the subject or user to complete the HAIL™ method and facilitate their emotional reframing. A practitioner may work with multiple subjects simultaneous as a group leader or proctor. If there are multiple subjects, the HAIL™ method may incorporate group or subject to subject interaction to further facilitate the performance of steps 120-150 and the emotional growth and development they provide. Alternatively, a practitioner may work with a single subject and assist through a more intimate conversational process. A practitioner and a subject may interact in person or indirectly through a computer or other device that facilitates long-distance communication.

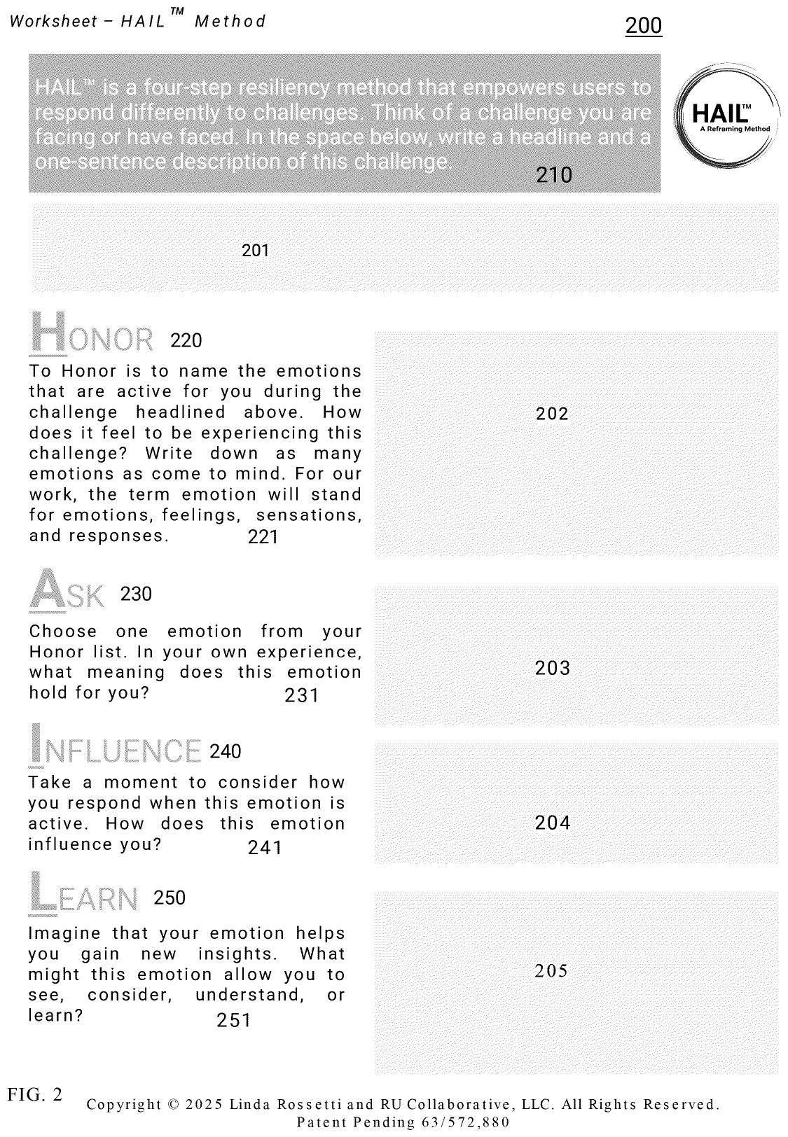

FIG. 2 is a graphical user interface (GUI) 200 utilized by an embodiment of the invention for facilitating a user's emotional reframing. While the HAIL™ method and certain embodiments of the disclosed methods and apparatuses can be practiced without any visual aid or corresponding apparatus, the use of such devices can be helpful for both the user and/or the practitioner. A GUI can comprise elements that are configured to both receive and display a user's responses to the questions asked during the HAIL™ method. Actively displaying those responses can assist the user to better explore and understand their analyzed emotions and responses, deepening the benefits provided by the resulting emotional reframing and developmental growth.

The GUI 200 can display elements 210, 220, 230, 240, 250 that correspond to the steps 110, 120, 130, 140, 150 of the HAIL™ method described in flow chart 100 of FIG. 1. Each element 210, 220, 230, 240, 250 may include corresponding prompts, questions, or other motivators that facilitates a viewer of the GUI 200 to complete or assist with completion of the HAIL™ method. Referring to the GUI 200, the Honor 220 element displays prompts 221, the Ask 230 element displays prompts 231, the Influence 240 element displays prompts 241, and the Learn 250 element displays prompts 251. Prompts 221, 231, 241, 251 are configured to guide and facilitate users of the HAIL™ method to complete their corresponding step. Prompts 221, 231, 241, 251 may be in written, video, audio or any other format. Prompts 221, 231, 241, 251 may be designed for a user to complete the HAIL™ method as an individual, in a group, or with a practitioner. GUI 200 may also include prompts or provide assistance for step 210 that facilitates a user to select a time period for which they want to apply the HAIL™ method. In alternative embodiments, step 110 may be completed by a user separately without element 210 or associated prompts. In additional alternative embodiments, the time period may be selected or assigned by individuals other than the user, for example a group seminar for college students focusing on the time period of transition after graduation or during matriculation.

Elements 210, 220, 230, 240, 250 may include editable sections and/or sub-elements 201, 202, 203, 204, 205. These sections 201, 202, 203, 204, 205 are configured to receive the time period selection and answers to prompts 221, 231, 241, 251 that guide a user through the HAIL™ method. As several steps of the HAIL™ method reference or incorporate responses to other methods, recoding those responses further enables a user to better understand their emotional and behavioral reactions. Furthermore, the act of preparing and recording an answer can assist a user to deepen their understanding of the self-exploration and emotional reframing induced by the HAIL™ method. Sections 201, 202, 203, 204, 205 can be configured to receive and display text, video, audio and/or any other forms of storing and/or displaying responses to prompts 221, 231, 241, 251. A user may provide their answers to prompts 221, 231, 241, 251 for recordation and display by sections 201, 202, 203, 204, 205 in any all possible manners or mediums. Sections 201, 202, 203, 204, 205 may be edited directly by a user or by a third party such as a practitioner.

Referring specifically to GUI 200, section 202, part of Honor element 220, is configured to receive and display emotions experienced by the user during a time period. The chosen time period may be displayed in section 201. Prompts 221 can be used to guide the user to identify and list those emotions. Section 203, part of Ask element 230, is configured to receive and display additional periods of time the user experienced a selected emotion. The selected emotion may be chosen from information displayed in section 202. Prompts 231 can be used to guide the user to identify and list those time periods. Section 204, part of Influence element 240, is configured to receive and display behaviors, internal and/or external, of the user associated with a selected emotion. Prompts 241 can be used to guide the user to identify and list those behaviors. Section 205, part of Learn element 250, is configured to receive and display effects of the selected emotion including any lessons, insights, or wisdom provided. Prompts 251 can be used to guide the user to identify and list those effects. When completed by the user, sections 201, 202, 203, 204, 205 facilitate practice of the HAIL™ method and the user's process of emotional reframing and developmental growth.

GUI 200 includes dedicated elements 210, 220, 230, 240, 250 and corresponding editable sections 201, 202, 203, 204, 205 for each step of the HAIL™ method. Alternative embodiments of the disclosed methods and apparatuses may utilize a GUI or multiple GUIs that have shared elements and/or sections for multiple steps of the HAIL™ method. In other embodiments, a single step of the HAIL™ method may have multiple elements and/or editable sections. As a non-limiting example, an editable section corresponding to each question of prompts 221, 231, 241, 251. The GUI may also provide a list of common emotions that a user may select from to complete element 202 of Honor element 220. Similar lists may be provided for the Ask element 230, Influence element 240, and/or Learn element 250 that permit a user to select from a set of pre-generated options. These lists may be especially useful when a user is less sophisticated or younger and needs further assistance in answering prompts 221, 231, 241, 251. Some embodiments of the disclosed methods and apparatuses may utilize customizable GUIs that can be adapted to particular users, facilitators, or settings.

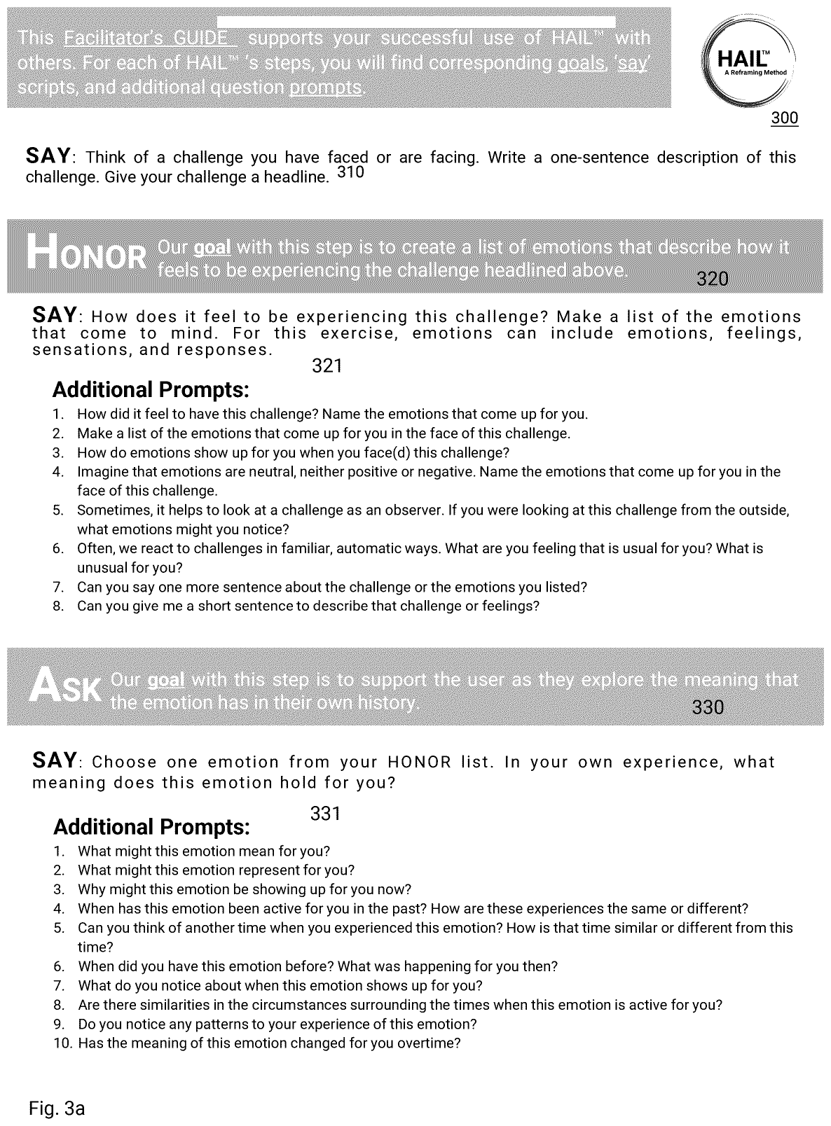

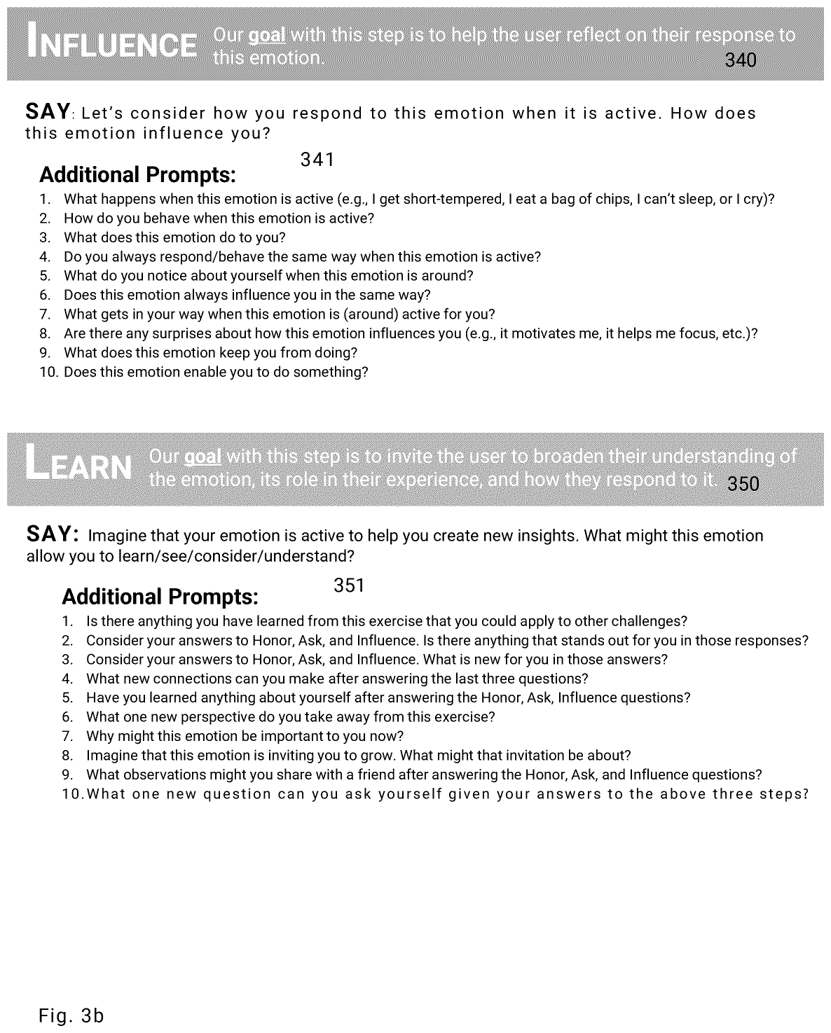

FIGS. 3a and 3b are the first and second page of a graphical user interface (GUI) 300 utilized by an embodiment of the invention for facilitating guidance by a practitioner of a subject's emotional reframing. In addition to or separate from the user presenting GUI 200, some embodiments of the disclosed methods and apparatuses utilize a practitioner GUI 300. The practitioner GUI 300 may be shared with the user focused GUI 200 and their specific elements can be interchanged and/or shared as desired. The practitioner GUI 300 includes a practitioner element that includes sections or sub-elements 310, 320, 330, 340, 350 that correspond to the steps of the HAIL™ method. One or more of the sections or sub-elements 310, 320, 330, 340, 350 can include a set of prompts 321, 331, 341, 351 that help facilitate the user or subject to perform the HAIL™ method. As shown in FIGS. 3a and 3b, sections 310, 320, 330, 340, 350 and/or prompts 321, 331, 341, 351 may include a description of the goal of the corresponding HAIL™ method step, an initial question, and additional prompts. GUI 300 and its displayed content can be used by a practitioner in combination with use of GUI 200 by a user to assist the user in responding to prompts 221, 231, 241, 251 and completing editable sections 201, 202, 203, 204, 205. Prompts 321, 331, 341, 351 may be in written, video, audio, or any other format. Prompts 321, 331, 341, 351 may be interactive or static. Prompts 321, 331, 341, 351 may be designed for a single subject and practitioner or a group environment.

In one embodiment of the invention, GUI 300 includes a practitioner element comprised of sections 310, 320, 330, 340, 350 and prompts 321, 331, 341, 351 are configured to be utilized by a practitioner to i) identify emotions experienced by the subject during a time period, ii) identify at least one additional period of time the subject experienced at least one emotion selected from the emotions experienced by the subject during the time period, iii) identify behaviors of the subject associated with the selected at least one emotion, and iv) identify effects of the selected at least one emotion. In additional embodiments GUI 300 may also include elements to be utilized by a practitioner to identify a time period during which the user experienced a challenge, transition, or similar disruption. In the same embodiment, elements 210, 220, 230, 240, 250 of GUI 200 can serve as a subject element configured to display: i) the emotions experienced by the subject during the time period, ii) at least one additional period of time the subject experienced the selected at least one emotion and the meaning that emotion holds in their experiences, iii) the behaviors of the subject associated with the selected at least one emotion, and iv) the effects of the selected at least one emotion. GUI 200 and GUI 300 may be a single GUI with both practitioner element and subject element or, in alternative embodiments, be separate GUIs. Both GUIs 200 and 300 may be utilized, together or individually, in an online, in person, or hybrid environment. GUIs 200 and 300 may be configured to display their component sections and/or content as worksheets or guides for use by a user and/practitioner during application of the HAIL™ method.

The HAIL™ method, when facilitated by a user utilizing GUI 200, a practitioner utilizing GUI 300, similar GUIs or apparatuses, and/or any combination thereof, improves the user's ability to understand and process the emotions experienced by the user during the selected time period. This includes, but is not limited to, providing a better understanding of their experienced emotions and being able to control their behavioral responses to the emotions and the circumstances that trigger them. These improvements correspond with the benefits provided by clinical behavioral therapy (CBT) despite the HAIL™ method being a non-clinical treatment. In other words, the HAIL™ method and embodiments of the disclosed methods and apparatuses are ways of increasing access to new and promising developments in mental health, developmental growth, and self-actualization. The HAIL™ method, when facilitated by embodiments of the disclosed methods and apparatuses and/or GUIs 200 and 300, has been found to a) generate greater resiliency in users, b) generate and/or make a user aware of a new behavioral response, c) improve a user's agency (agency is defined as a person's ability to act on their own behalf, d) create a new way of approaching challenges for a user, and e) generate a new perspective on circumstances and challenges encountered by a user.

Further included in this disclosure are methods of training and/or teaching users to practice the HAIL™ method and practitioners to assist users to practice the HAIL™ method. These methods can be performed in a group setting and in person, online, or hybrid environment. These embodiments may utilize GUIs 200, 300 and/or similar apparatuses and devices.

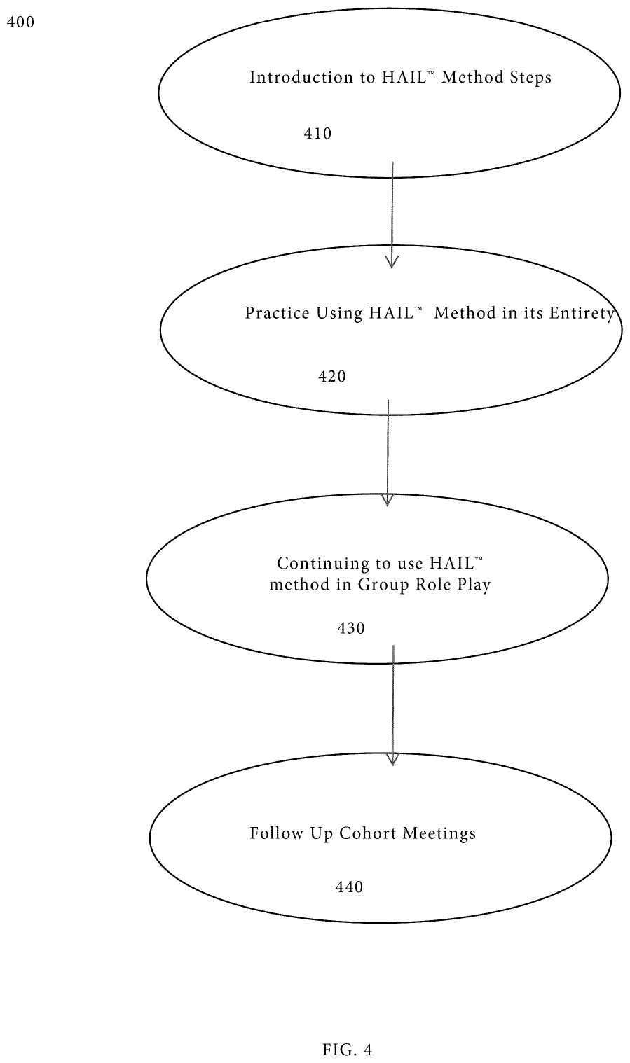

FIG. 4 is a flow chart 400 of an embodiment of the invention enabling practice of the HAIL™ method. One of the key benefits of the HAIL™ method and embodiments of the disclosed methods and apparatuses is their ability to be applied by ordinary people increasing access to valuable developmental growth and emotional reframing tools. Through dedicated training, people are able to learn how to both utilize the HAIL™ method as a user and apply the HAIL™ method as a practitioner. Flow chart 400 describes an example embodiment for teaching the HAIL™ method.

In step 410, the HAIL™ method and its component steps, Honor, Ask, Influence, and Learn are introduced. Step 410 may utilize both direct teaching and collaborative group work. Additionally, learning tools, such as but not limited to GUIs 200 and 300 may be used to introduce the HAIL™ method and its component steps. Students may take turns being both a subject and a practitioner and explore how both roles interact with and utilize the Honor, Ask, Influence, and Learn steps.

In step 420, students practice using the HAIL™ method in its entirety from start to finish. Students may be broken into pairs where each person takes a turn practicing the entire 4-step method as a subject and as a practitioner. This practice may utilize GUIs 200 and 300 or other tools, worksheets, or guides related for utilizing the HAIL™ method. New pairs may be created so that the students have the opportunity to learn how different individuals interact with the HAIL™ method. Supplemental reflection periods during which the students answer query forms that explore self-assessment and identify opportunities for further study may also be included in second step 420.

In step 430, the students continue to use and practice the HAIL™ method in group role play. This may comprise three-person break-out groups, where one person takes on the role of subject/user, one person takes the role of practitioner, and one person takes the role of an observer. All group members share their observations, impressions, and insights provided by their role. This debriefing may take place both within the smaller break-out groups and/or in a single large group of all the students. During third step 430, roles may be rotated, and break-out groups reformed to facilitate additional practice and insights.

In step 440, students attend at least one follow up meeting to discuss how to introduce and utilize the HAIL™ method in their day-to-day activities and review the approved applications of the HAIL™ method and the value it provides.

Methods of teaching the HAIL™ method may also include supplemental steps in addition to steps 410, 420, 430, 440 of flow chart 400 including but not limited to lectures, teacher demonstrations, written materials and guides, video examples, interactive demonstratives, group reflections and discussions, homework worksheets, testing, discussion groups, and other known teaching tools.

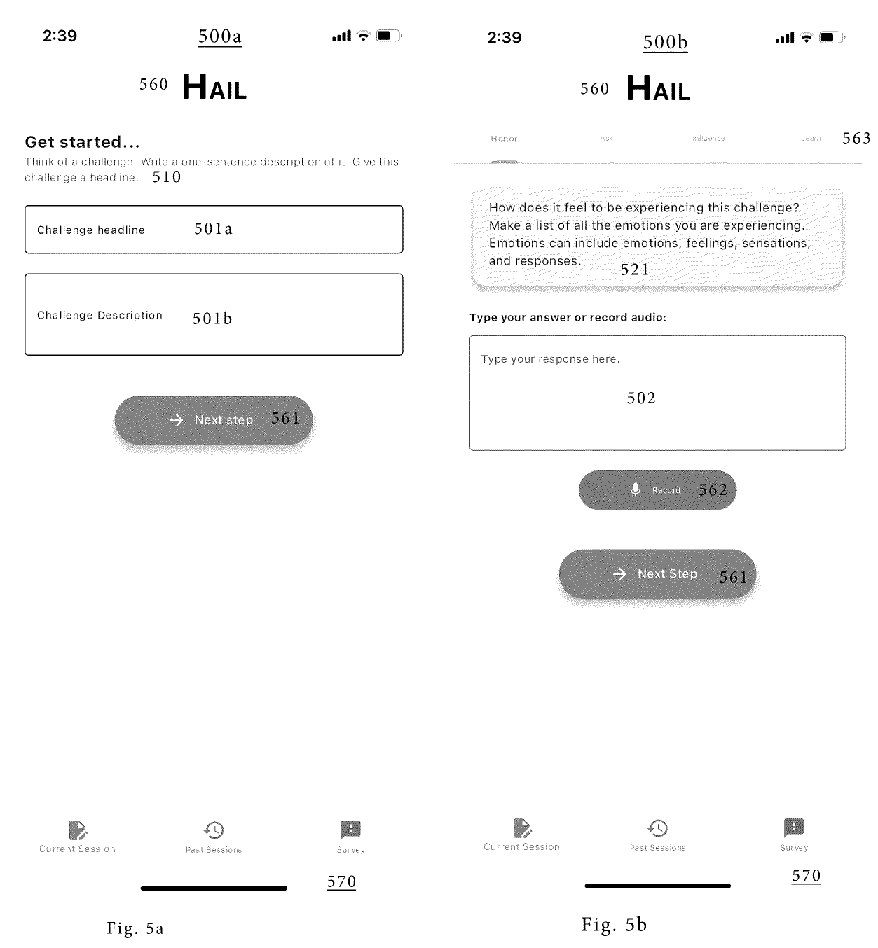

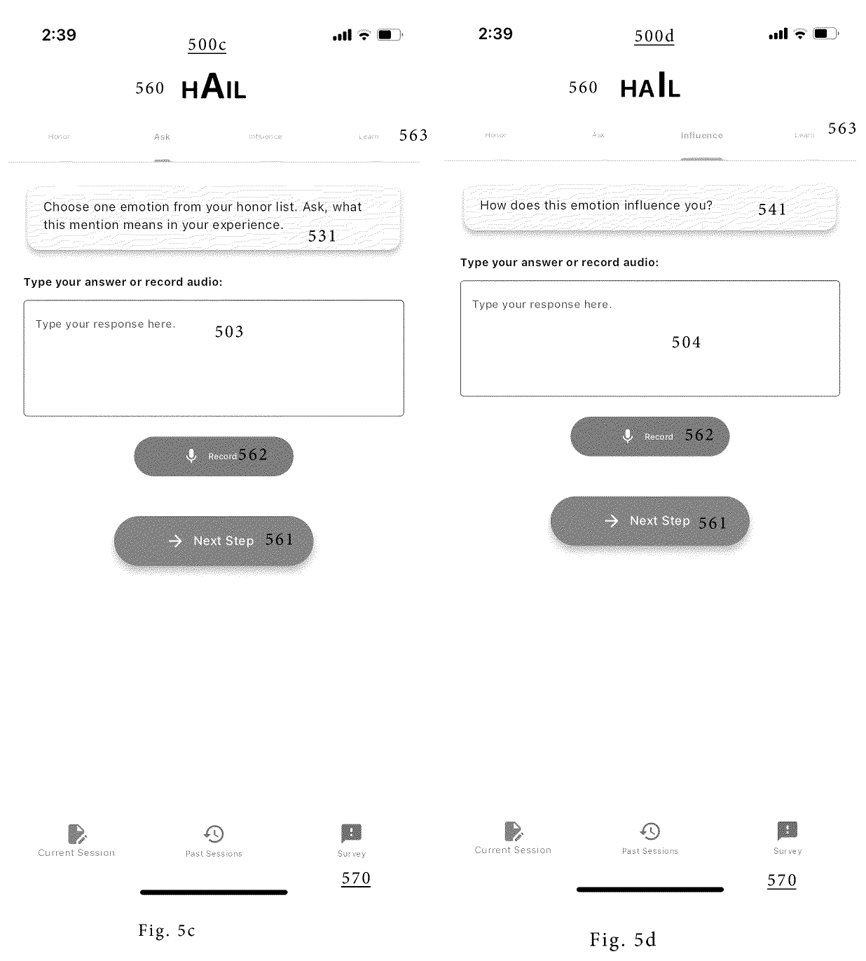

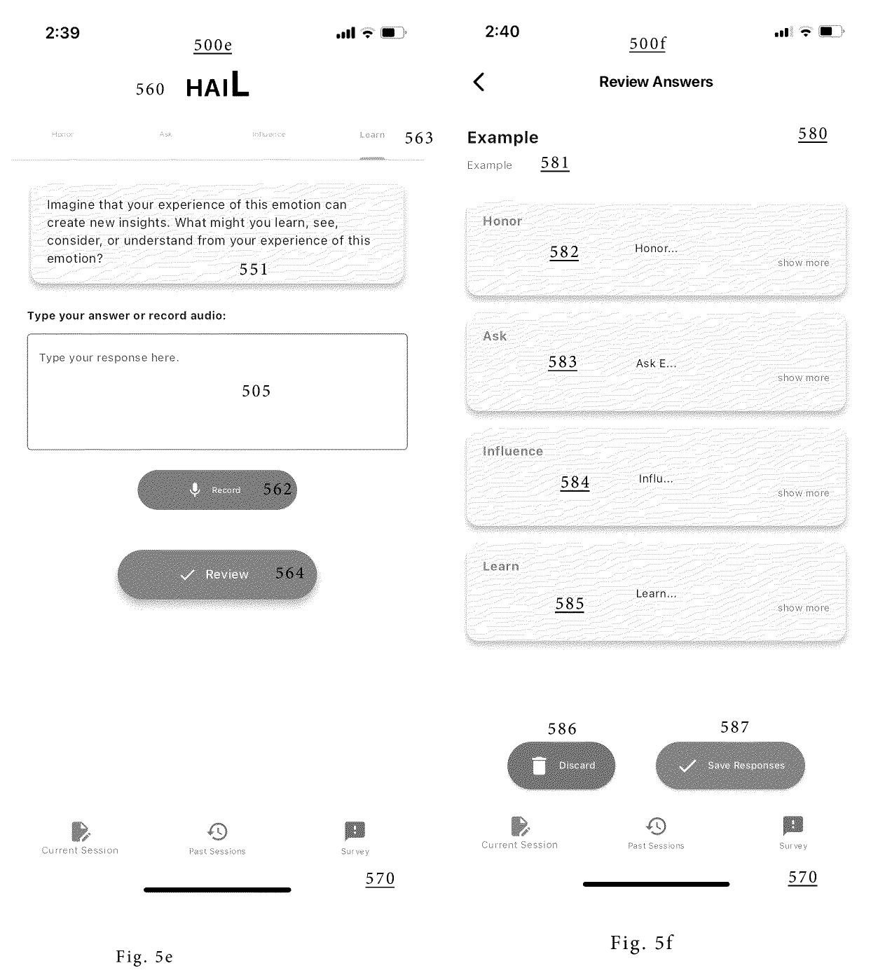

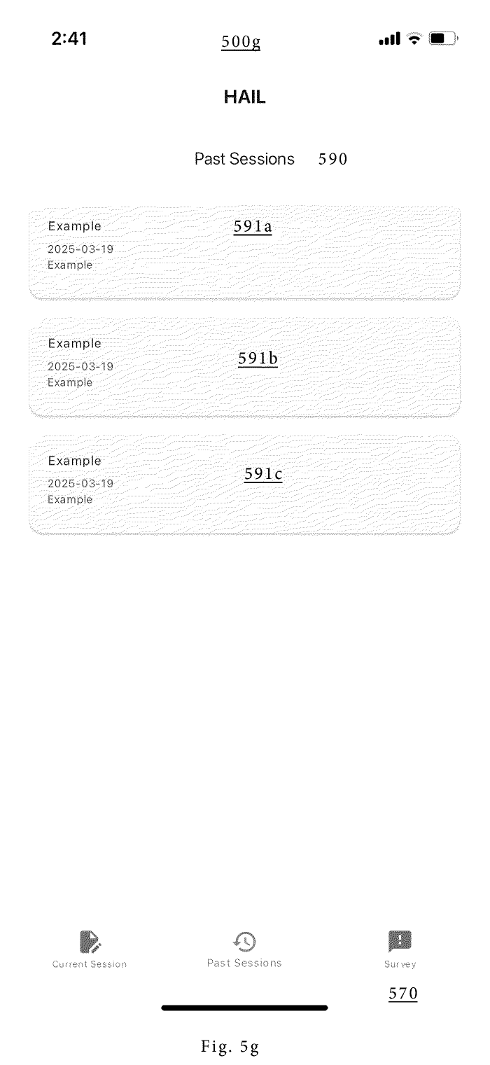

FIGS. 5a-g are screenshots of an App (“HAIL™ App”) configured generate and display a graphical user interface (GUI) 500a-g utilized by an embodiment of the invention for facilitating a user's emotional reframing. The graphical user interface may be comprised of multiple different components 500a-g. FIGS. 5a-g show an example of GUI components 500a-g generated and displayed by a smartphone operating the HAIL™ App but one skilled in the art would be aware that they could be similarly utilized by other forms of computing devices. The GUI components 500a-g may be displayed on any desired combination of screens, devices, or components capable of interfacing with the HAIL™ App. The GUI components 500a-g can be generated based on computer readable instructions stored on the memory of a computing device accessible using the computing device operating the HAIL™ App. The GUI components 500a-g can utilize input/output devices operated by at least one computing device, e.g., smartphone operating the HAIL™ App, to receive inputs from users and display GUI components 500a-g.

The GUI components 500a-g may include a HAIL title 560, header 563, and/or footer 570 to facilitate a user navigating between the GUI components. The GUI components 500a-g (collectively GUI 500) may also include a “next step” button 561 to enable a user to proceed sequentially through steps of the HAIL™ method using the displayed GUI and its components 500a-g. The components 500a-g of the GUI can include any number of individual or shared elements (e.g., header 563 and footer 570).

GUI 500 can be used to practice the HAIL™ method 100. Component 500a, may be displayed on initial starting screen(s) viewed by a practitioner or user of the HAIL™ method 100. Component 500a displays a starting prompt 510 which requests that the practitioner identify a challenge. Component 500a includes input elements 501a, 501b to allow the practitioner, user, or both to respond to the starting prompt 510. Component 500a, and other components of GUI 500, may include any number of input elements to allow a practitioner, user, or both to provide the requested information and organize that information in any desired manner. Next step button 561 can be pressed by a practitioner, user, or both to move to the next component 500b.

Components 500b-e correspond with the “Honor” 120 (component 500b), “Ask” 130 (component 500c), “Influence” 140 (component 500c), and “Learn” 150 (component 500d) steps of the HAIL™ method 100. Component 500b, displays honor prompt 521 and receives a user's response in input element 502. By answering honor prompt 521, a practitioner or user follows or facilitates step 120 of the HAIL™ method 100. Component 500c, displays ask prompt 531 and receives a user's response in input element 503. By answering ask prompt 531, a practitioner or user follows or facilitates step 130 of the HAIL™ method 100. Component 500d, displays influence prompt 541 and receives a user's response in input element 504. By answering influence prompt 541, a practitioner or user follows or facilitates step 140 of the HAIL™ method 100. Component 500e, displays learn prompt 551 and receives a user's response in input element 505. By answering influence prompt 551, a practitioner or follows or facilitates step 150 of the HAIL™ method 100. Prompts 521, 531, 541, 551 can include prompts 221, 321, 231, 331, 241, 341, 251, and 351 of FIGS. 2, 3a, and 3b. Components 500b-e may be configured to display alternative or additional prompts 521, 531, 541, 551 for use by a practitioner or user based on a practitioner's request, user's input, or other properties to enable the HAIL™ method to be customized to a particular setting or implementation. Prompts 521, 531, 541, 551 may be displayed in text, audio, video, or other format.

Input elements 501a, 501b, 502, 503, 504, and 505 can receive and cause the provided response to be stored in a computing devices memory, cloud, or other storage device. Input elements 501a, 501b, 502, 503, 504, and 505 can receive a practitioner's or user's response in text or in audio through use of the record button 562. Next step button 561 can be pressed by a practitioner or user to move on to the next step of the HAIL™ method 100 and cause the GUI 500 to display the corresponding component 500b-e. Alternatively, header 563 can be used to navigate directly to a specific step corresponding to the word of the HAIL™ method 100. In components 500b-e the HAIL title 560 emphasizes the corresponding letter of the HAIL™ method, e.g., in component 500b the “H” is emphasized. Header 563 includes the steps of the HAIL™ method and a practitioner or user can interact with each step to cause the respective GUI component 500b-e to be displayed.

Component 500e includes a review button 564 in place of the next step button 561. Interacting with review button 564 causes review component 500f to be generated and displayed. Review component 500f includes a summary 580 of the answers provided by the practitioner or user in response to prompts 521, 531, 541, 551. Elements 581-585 display the information entered into input elements 501-505 respectively to allow for comprehensive review and revision. The discard button 586 allows a practitioner or user to discard the aforementioned information while the save responses button 587 allows a user to save aforementioned information. Saved information, can be accessed in the archive component 500g that includes a list of past session 590 with the saved information 591a-c. A practitioner or user can use footer 570 to navigate between the archive component 500g and the active session utilizing components 500a-g.

GUI 500 and the software that generates it may be associated with a particular user or practitioner. Information, such as the responses saved using save responses button 587, can be similarly associated with a particular user or practitioner to allow for easier or selective access.

GUI 500, the HAIL™ App, and other embodiments of the invention can be utilized to practice or facilitate practice of the HAIL™ method 100 in a variety of scenarios and combinations. As non-limiting examples, a practitioner may use GUI 500 alone to facilitate application of the HAIL™ method 100 by a user, both a practitioner and a user can use GUI 500 (either a shared copy or individual copies) to facilitate application of the HAIL™ method 100 by a user, a user may use GUI 500 alone, to apply the HAIL™ method 100, with our without a facilitating practitioner, and a practitioner or user can use GUI 500 to learn or teach the HAIL™ method 100. A user and a practitioner may exchange or rotate roles and use GUI 500, the HAIL™ App, and other embodiments of the invention in any desired application or scenario.

### Digital Processing Environment

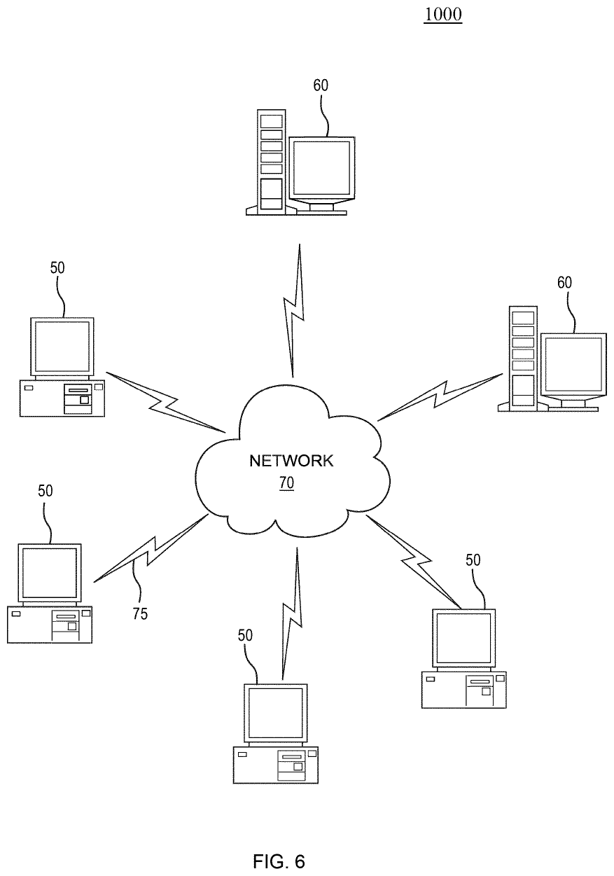

FIG. 6 illustrates a computer network or similar digital processing environment in which the present embodiments 1000 may be implemented. Client computer(s)/devices 50 and server computer(s) 60 provide processing, storage, and input/output devices executing application programs and the like. Client computer(s)/devices 50 can also be linked through communications network 70 to other computing devices, including other client devices/processors 50 and server computer(s) 60. Communications network 70 can be part of a remote access network, a global network (e.g., the Internet), cloud computing servers or service, a worldwide collection of computers, Local area or Wide area networks, and gateways that currently use respective protocols (TCP/IP, Bluetooth, etc.) to communicate with one another. Other electronic device/computer network architectures are suitable.

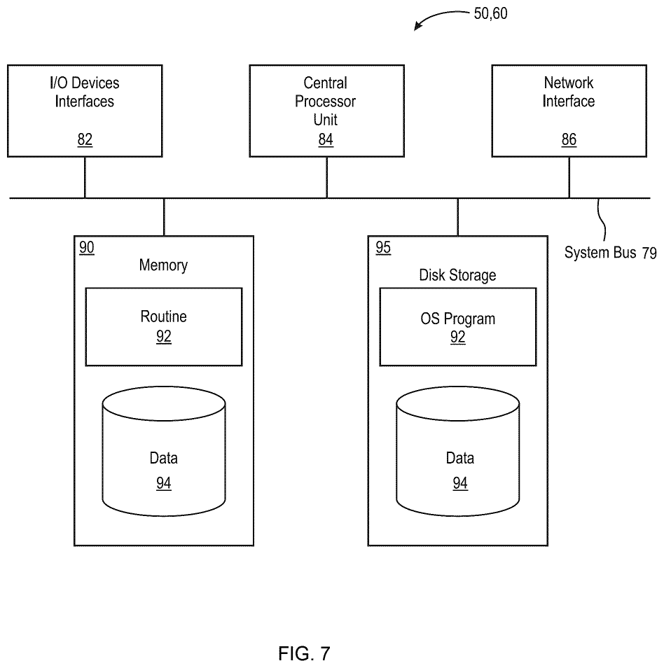

FIG. 7 is a diagram of the internal structure of a computer (e.g., client processor/device 50 or server computers 60) in the computer system of FIG. 6. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, and network ports) that enables the transfer of information between the elements. Attached to system bus 79 is I/O device interface 82 for connecting various input and output devices (e.g., keyboard, mouse, displays, printers, and speakers) to the computer 50, 60. Network interface 86 allows the computer to connect to various other devices attached to a network (e.g., network 70 of FIG. 6). Memory 90 provides volatile storage for computer software instructions 92 and data 94 used to implement embodiments (e.g., GUIs 200, 300, 500, their component elements, information received or submitted through GUIs 200, 300, 500, and any other interfaces, tools, worksheets, demonstrations, or guides utilized by a user to facilitate practice of the HAIL™ method or utilized by a practitioner to assist a subject to practice the HAIL™ method including, but not limited to, the HAIL™ App). Memory 90 may securely store information locally on a specified device and protect that stored information from access by or using a network (e.g., network 70 of FIG. 6). Disk storage 95 provides non-volatile storage for computer software instructions 92 and data 94 used to implement many embodiments. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, cloud storage, and flash drives) that provides at least a portion of the software instructions for the system. Computer program product 92 can be installed by any suitable software installation procedure, as is well known in the art. In another embodiment, at least a portion of the software instructions may also be downloaded over a cable, communication, and/or wireless connection. In other embodiments, the programs are a computer program propagated signal product 75 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product. In general, the term “carrier medium” or transient carrier encompasses the foregoing transient signals, propagated signals, propagated medium, storage medium and the like. In other embodiments, the program product 92 may be implemented as a so-called Software as a Service (SaaS), or other installation or communication supporting end-users.

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of facilitating emotional reframing, comprising:
obtaining, a time period;
identifying emotions experienced by a person during the time period;
receiving, a selection of at least one emotion from the emotions experienced by the person during the time period;
identifying a meaning held by the person associated with the selected at least one emotion;
identifying behaviors of the person associated with the selected at least one emotion; and
identifying effects of the selected at least one emotion facilitating improvement by the person to understand, process, or alter behaviors related to the emotions experienced by the person during the time period.

2. The method of claim 1 wherein the time period includes a challenge experienced by the person.

3. The method of claim 1 wherein a practitioner performs or assists the person to perform at least one of i) obtaining a time period, ii) identifying the emotions, iii) receiving the selection of at least one emotion, iv) identifying the meaning held by the person, v) identifying the behaviors of the person, and vi) identifying effects of the selected at least one emotion.

4. The method of claim 3 further comprising:
generating a graphical user interface (GUI), the graphical user interface (GUI) configured to display:
at least one prompt element comprising prompts for use by the practitioner; and
at least one input element configured to receive responses provided by the person.

5. Wherein the improvement by the person to understand, process, or alter behaviors related to the emotions experienced by the person during the time period includes at least one of an ability to handle failure, an ability to deal with difficulties, an ability to handle unpleasant feelings, an ability to be aware of multiple options, an ability to address new challenges.

6. An apparatus for facilitating a user's emotional reframing, comprising:
a processor and one or more storage devices storing instructions that are operable, when executed by the processor, to generate a graphical user interface (GUI) configured to display:
a first element configured to receive and display emotions experienced by a user during a time period;
a second element configured to receive and display a meaning held by the user associated with at least one emotion selected from the emotions experienced by the user during the time period;
a third element configured to receive and display behaviors of the user associated with the selected at least one emotion; and
a fourth element configured to receive and display effects of the selected at least one emotion,
wherein the graphical user interface (GUI) facilitates improvement of the user to understand, process, or alter behaviors related to the emotions experienced by the user during the time period.

7. The apparatus of claim 6 wherein the processor and one or more storage devices is one of a smartphone, computer, or tablet.

8. The apparatus of claim 6 wherein:
At least one of the first element, the second element, the third element, and the fourth element comprises:
at least one prompt element configured to display, to the user, prompts; and
at least one input element configured to receive, from the user, responses to the displayed prompts.

9. The apparatus of claim 8 wherein the received responses to the displayed prompts facilitates improvement of the user to understand, process, or respond differently to the emotions experienced by the user during the time period.

10. The apparatus of claim 8 wherein the graphical user interface (GUI) is further configured to display a summary element displaying the received responses to the displayed prompts.

11. An apparatus for facilitating emotional reframing guidance, comprising:
a processor and one or more storage devices storing instructions that are operable, when executed by the processor, to generate a graphical user interface (GUI) configured to display:
at least one prompt element configured to display conversational prompts, the conversational prompts configured to be utilized to enable a subject to: i) identify emotions experienced by the subject during a time period, ii) identify a meaning held by the subject associated with at least one emotion selected from the emotions experienced by the subject during the time period, iii) identify behaviors of the subject associated with the selected at least one emotion, and iv) identify effects of the selected at least one emotion; and
at least one display element configured to display: i) the emotions experienced by the subject during the time period, ii) the at least one additional period of time the subject experienced the selected at least one emotion, iii) the behaviors of the subject associated with the selected at least one emotion, and iv) the effects of the selected at least one emotion,
wherein the graphical user interface (GUI) facilitates improvement of the subject to understand, process, and alter behaviors related to the emotions experienced by the subject during the time period.

12. The apparatus of claim 11 wherein the processor and one or more storage devices is one of a smartphone, computer, or tablet.

13. The apparatus of claim 11 wherein the graphical user interface (GUI) is further configured to display at least one input element configured to receive responses to the conversational prompts.

14. The apparatus of claim 13 wherein the at least one input element is configured to receive responses to the conversational prompts by text, audio, or visual methods.

15. The apparatus of claim 13 wherein the at least one subject element is further configured to display the received, responses to the conversational prompts by text, audio, or visual methods.

16. A method of facilitating a user's emotional reframing through use of a processor and one or more storage devices configured to store instructions that are operable when executed by the processor to generate a graphical user interface (GUI), the method comprising:
receiving and displaying, by a first element of the GUI, emotions experienced by the user during a time period;
receiving and displaying, by a second element of the GUI, a meaning associated with at least one emotion selected from the emotions experienced by the user during the time period;
receiving and displaying, by a third element of the GUI, behaviors of the user associated with the selected at least one emotion; and
receiving, and displaying, by a fourth element of the GUI, effects of the selected at least one emotion,
wherein the graphical user interface (GUI) facilitates improvement of a user to understand, process, or alter behaviors related to the emotions experienced by the user during the time period.

17. The method of claim 16 wherein graphical user interface (GUI) generated by a smartphone application.

18. The method of claim 16 wherein:
at least one of the first element, the second element, the third element, and the fourth element comprises:
at least one prompt element configured to display prompts; and
at least one input element configured to receive responses to the displayed prompts.

19. The method of claim 18 wherein at least one of the emotions experienced by the user during a time period, a meaning associated with the at least one emotion selected from the emotions experienced by the user during the time period, behaviors of the user associated with the selected at least one emotion, and effects of the selected at least one emotion comprise the received responses to the displayed prompts.

20. The method of claim 18 further comprising storing, in the one or more storage devices the received responses to the displayed prompts.
